(19) United States
(12) Patent Application Publication
(10) Pub. No.: US 2024/0317659 A2
(43) Pub. Date: Sep. 26, 2024

(54) PROCESS FOR THE PRODUCTION OF GLYCOLS

(71) Applicant: SHELL OIL COMPANY, HOUSTON, TX (US)

(72) Inventors: Pieter HUIZENGA, Amsterdam (NL); Eugene VAN KRUCHTEN, Amsterdam (NL); Karin BUS, Amsterdam (NL)

(21) Appl. No.: 18/245,572

(22) PCT Filed: Oct. 4, 2021
(86) PCT No.: PCT/EP2021/077283

§ 371 Date: Mar. 16, 2023

(30) Foreign Application Priority Data
Oct. 7, 2020 (EP) 20200630.0

(51) Int. Cl. C07C 29/84; C07C 29/17; C07C 29/82

(52) U.S. Cl. CPC C07C 29/84; C07C 29/172; C07C 29/82

(57) ABSTRACT

The invention provides a process for the separation of a diol from a product stream. The process includes the steps of: i) separating the product stream comprising three or more C2 to C6 diols, C3 to C6 sugar alcohols, and C4 to C6 polyhydric alcohols with at least 3 hydroxyl groups in the molecule, and a catalyst, to produce a first stream comprising the three or more C2 to C6 diols; ii) separating the first stream comprising the three or more C2 to C6 diols into a) a second stream comprising a first diol and unsaturated hydrocarbons and/or one or more compounds with a carbonyl group and b) a third stream comprising two or more diols; iii) hydrogenating the second stream comprising a first diol and unsaturated hydrocarbons and/or one or more compounds with a carbonyl group to provide a purified diol stream.

### FIELD OF THE INVENTION

The invention relates to a purification process for preparing diols which meet product specifications for color bodies and transmission of radiation, more particularly for preparing monoethylene glycol (MEG) meeting the product specifications on color bodies and transmission of radiation.

### BACKGROUND

Ethylene glycol and propylene glycol are valuable materials with a multitude of commercial applications, e.g. as heat transfer media, antifreeze, and precursors to polymers, such as polyethylene terephthalate (PET). Ethylene and propylene glycols are typically made on an industrial scale by hydrolysis of the corresponding alkylene oxides, which are the oxidation products of ethylene and propylene, produced from fossil fuels. Ethylene glycol has a wide range of uses, including a very important use as the basic raw material for producing the polyester of polyester fibers, ethylene glycol here being generally referred to as the fiber-grade ethylene glycol product.

In recent years, increased efforts have focused on producing chemicals, including glycols, from renewable feedstocks, such as sugar-based materials. Current methods for the conversion of saccharides to glycols revolve around a hydrogenation/hydrogenolysis process as described in Angew. Chem. Int. Ed. 2008, 47, 8510-8513. For example, U.S. 2011/312050 describes a continuous process for the catalytic generation of polyols from cellulose, in which the cellulose is contacted with hydrogen, water and a catalyst to generate an effluent stream comprising at least one polyol.

CN 102643165 is directed to a catalytic process for reacting sugar in an aqueous solution with hydrogen in the presence of a catalyst in order to generate polyols.

As with many chemical processes, the product stream in these reactions comprises a number of desired materials, diluents, by-products and other undesirable materials. In order to provide a high value process, the desirable product or products must be obtainable from the product stream in high purity with a high percentage recovery of each product and with as low as possible use of energy and complex equipment.

One index for measuring the quality of the fiber-grade ethylene glycol products is the UV-light transmittance at 220 nm, because this will affect the luster and chrominance of the downstream polyester products. The prior art teaches using ion exchange resin as the catalyst to refine and purify ethylene glycol, e.g., U.S. Pat. No. 6,242,655 describes a method of using a strongly acidic cation exchange resin as the catalyst, wherein after the treatment, the content of the aldehyde group in the ethylene glycol products decreases from 20 ppm to 5 ppm or less. However, the defect of the existing method is that the content of the aldehyde group in the ethylene glycol products can only be removed to about 2 ppm at most, but the UV-light transmittance at 220 nm of the ethylene glycol products at this moment still does not reach a very ideal value.

Therefore, it would be advantageous to improve the UV-light transmittance of the carbohydrate-based ethylene glycol products and further guarantee the quality of the products. An improved method suitable for the recovery of high purity glycols, preferably meeting the UV-light transmittance standards is needed.

### SUMMARY OF THE INVENTION

The invention provides a process for the separation of a diol from a product stream. The process includes the steps of: i) separating the product stream comprising three or more C2 to C6 diols, C3 to C6 sugar alcohols, and C4 to C6 polyhydric alcohols with at least 3 hydroxyl groups in the molecule, and a catalyst, to produce a first stream comprising the three or more C2 to C6 diols; ii) separating the first stream comprising the three or more C2 to C6 diols into a) a second stream comprising a first diol and unsaturated hydrocarbons and/or one or more compounds with a carbonyl group, and b) a third stream comprising two or more diols; iii) hydrogenating the second stream comprising a first diol and unsaturated hydrocarbons and/or one or more compounds with a carbonyl group to provide a purified diol stream.

### BRIEF DESCRIPTION OF THE DRAWINGS

The process of the present invention will be better understood by referring to the following detailed description of preferred embodiments and the drawings referenced therein, in which:

FIG. 1 illustrates a block flow diagram of an embodiment of a process of the present invention;

FIG. 2 illustrates a block flow diagram of an alternative embodiment of a process of the present invention.; and

FIG. 3 illustrates the UV transmission specification against embodiments of the invention.

### DETAILED DESCRIPTION OF THE INVENTION

The following description of the variations is merely illustrative in nature and is in no way intended to limit the scope of the disclosure, its application, or uses. The description and examples are presented herein solely for the purpose of illustrating the various embodiments of the disclosure and should not be construed as a limitation to the scope and applicability of the disclosure.

The terminology and phraseology used herein is for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited.

Also, as used herein any references to “one embodiment” or “an embodiment” means that a particular element, feature, structure, or characteristic described in connection with the embodiment is included in at least one embodiment. The appearances of the phrase “in one embodiment” in various places in the specification are not necessarily referring to the same embodiment.

Embodiments herein describe methods for the separation of a diol from a product stream. In some embodiments, the product stream is derived from a carbohydrate hydrogenolysis process. Such a product stream from a process for the hydrogenolysis of a carbohydrate-containing feedstock comprises certain desirable diols as well as by-products comprising diols and other materials. Carbohydrates include saccharides in including monosaccharides like e.g. glucose, fructose, xylose, di-saccharides like sucrose and polysaccharides like starch, cellulose and hemicellulose.

The product stream may have three or more C2 to C6 diols. Preferably, the separated diol and the three or more C2 to C6 diols in the product stream are selected from the group consisting of C2 to C6 glycols. The term glycol as used herein is given its usual meaning, i.e. a diol in which the two hydroxyl groups are present on vicinal carbon atoms. Preferably, the diol is monoethylene glycol (MEG) and the product stream comprises MEG and 1,2-butanediol (1,2-BDO), or the diol is monopropylene glycol (MPG) and the product stream comprises MPG and 2,3-pentanediol. Most preferably, the diol is monoethylene glycol (MEG) and the product stream comprises MPG and 1,2-butanediol (1,2-BDO). The product stream may also include three or more C2 to C6 diols and one or more components selected from the group of C3 to C6 sugar alcohols and C4 to C6 polyhydric alcohols with at least 3 hydroxyl groups in the molecule, and optionally a catalyst.

The three or more C2 to C6 diols product stream may be derived from any diol process such as, but not limited to, an oil route, i.e., direct hydration or process hydration, the hydrogenation of oxalate, or the hydrogenolysis of a carbohydrate-containing feedstock. In other embodiments, the product stream may be any diol stream that does not meet product specifications on color bodies and transmission of radiation.

Typically, the product stream from a process for the hydrogenolysis of a carbohydrate-containing feedstock comprises, as diols, at least MEG, MPG and 1,2-BDO. Other diols, such as 2,3-BDO, pentanediols, hexanediols and heptanediols may also be present.

As well as the three or more C2 to C6 diols, the product stream from hydrogenolysis reactions of carbohydrates may comprise oxygenates, hydrocarbons, catalyst, degradation products, and gases in any composition. The variety of compounds and their concentration depend on the carbohydrate-containing feedstock and the various hydrogenation and hydrogenolysis conversion conditions, including catalysts, reaction conditions such as temperature, pressure and carbohydrate concentration.

In one embodiment, the product stream comprises at least a mixture comprising MEG and 1,2-BDO. Other materials, such as MPG and other light glycols may be present in the mixture comprising MEG and 1,2-BDO. In this embodiment, the mixture comprising MEG and 1,2-BDO preferably has a weight ratio of MEG:1,2-BDO of at least 3:2. More preferably the weight ratio of MEG:1,2-BDO is at least 5:1. Most preferably the weight ratio of MEG:1,2-BDO is at least 20:1.

In another embodiment, the product stream comprises at least a mixture comprising MPG and 2,3-pentanediol. Other materials, such as light glycols may be present in the mixture comprising MPG and 2,3-pentanediol. In this embodiment, the mixture comprising MPG and 2,3-pentanediol preferably has a weight ratio of MPG:2,3-pentanediol of at least 3:2. More preferably the weight ratio of MPG:2,3-pentanediol is at least 5:1. Most preferably the weight ratio of MPG:2,3-pentanediol is at least 20:1.

In one embodiment, a process is described for the separation of a diol from the above described product stream. The process includes the steps of: (i) separating the product stream comprising three or more C2 to C6 diols, C3 to C6 sugar alcohols, and C4 to C6 polyhydric alcohols with at least 3 hydroxyl groups in the molecule, and a catalyst, to produce a first stream comprising the three or more C2 to C6 diols; (ii) separating the first stream comprising three or more C2 to C6 diols into a) a second stream comprising a first diol and unsaturated hydrocarbons and/or one or more compounds with a carbonyl group, and b) a third stream comprising two or more diols; (iii) hydrogenating the second stream comprising a first diol and unsaturated hydrocarbons and/or one or more compounds with a carbonyl group to provide a high purity diol stream. In some embodiments, the high purity diol meets product specification on color bodies and transmission of radiation.

Preferably, the separating step (i) of the above process is an evaporation step (i).Preferably, the evaporation step includes providing the product stream to a distillation column or flashing unit. The separation step provides a first stream including the three or more C2 to C6 diols. In some embodiments, where the separating step is an evaporation step (i), evaporation may occur at temperatures ranging from about 120 to about 250° C., preferably at temperatures ranging from about 150 to about 230° C., and more preferably at temperatures ranging from about 180 to about 210° C., most preferably at a temperature lower than 200° C., measured as the temperature of the bulk liquid in the reboiler (bottom of the column). In some embodiments, the evaporation step (i) may occur at pressures ranging from about 0.1 to about 2000 kPa. In some embodiments, the evaporation step (i) may have a number of theoretical stages, which varies in the range of from about 1 to about 140, or may be a flash vessel without trays or packing, which may be equipped with a demister to remove droplets entrained in the vapor phase.

The separation step (ii) of the above process includes separating the first stream into a second stream and a third stream. The second stream includes a first diol and unsaturated hydrocarbons and/or one or more compounds with a carbonyl group and the third stream includes two or more diols.

The separation step (ii) may occur in one or two distillation columns. In an embodiment that utilizes a single distillation column, the single distillation column operates at a temperature in the range of from 100 to 300° C. and a pressure in the range of from 0.1 to 2000 kPa to produce the second stream as a bottom stream including the first diol and unsaturated hydrocarbons and/or one or more compounds with a carbonyl group and the third stream as a top stream including two or more diols are removed from the distillation column. In some embodiments, the third stream may be an azeotrope of MEG and 1,2-BDO or an azeotrope of MPG and 2,3 BDO. The second stream would be sent to the hydrogenating step (iii) disclosed above. The third stream is subjected to one or more fractional distillation steps in order to produce desired products as pure product streams.

The single distillation column may be any suitable sort of column known in the art and may be equipped with trays or structured or unstructured packing. The number of theoretical stages may vary in the range of from 3 to 140 and may easily be determined by the skilled person on the basis of simple economic optimization experiments.

In an embodiment that utilizes two distillation columns, the first stream of three or more C2 to C6 diols is provided to a first distillation column, which is an extractive distillation column. The extractive distillation column may be any suitable sort of column known in the art and may be equipped with trays or structured or unstructured packing. The number of theoretical stages may vary in the range of from 3 to 140 and may easily be determined by the skilled person on the basis of simple economic optimization experiments.

An extractant is fed to the extractive distillation at or above the location where the first stream is provided. Preferably, the extractant is provided at the top of or a few stages below the top of the first distillation column.

The extractant is selected from the group of C3 to C6 sugar alcohols, C4 to C6 polyhydric alcohols with at least 3 hydroxyl groups in the molecule, and mixtures thereof. Sugar alcohols have the general formula HOCH2(CHOH)nCH2OH. Suitable sugar alcohols include glycerol, erythritol, threitol, arabitol, xylitol, ribitol, mannitol, sorbitol, galactitol and iditol. Although some of these sugar alcohols may be solid at room temperature, pressures and compositions for suitable extractant mixtures, they can be used as liquids at suitable temperatures and pressures in the process of the invention. In an embodiment, the extractant is glycerol which may be at least 50 w/w % of the extractant feed mixture into the distillation column.

Polyhydric alcohols that may be used as extractants include butanetriols, pentane triols, pentane tetraols, hexane triols, hexane tetraols, and hexane pentols.

Preferably, the extractant is added in an amount such that the weight ratio of the feed comprising extractant to the mixture comprising the at least one C2 to C7 diols is from about 1:2 to about 20:1. In some embodiments, the weight ratio of the feed comprising extractant to the mixture comprising the at least one C2 to C7 diols is at least 0.05:1, more preferably at least 0.1:1, even more preferably at least 0.25:1, based on the overall weight of the feed/mixture. Preferably, the weight ratio of the feed comprising the extractant to the first mixture comprising the at least one C2-C7 diols is at most 10:1, more preferably at most 5:1, even more preferably 2:1, more preferably at most 1.5:1, based on the overall weight of the feed/mixture.

The extractive distillation in the first distillation column is carried out at a temperature in the range of from 50 to 300° C., preferably of from 100 to 250° C. and at a pressure in the range of from 0.1 to 2000 kPa. Generally, a pressure of at least 1 kPa is preferred for economic reasons, with a pressure of at least 5 kPa more preferred for the same reasons. The pressure is at most 2000 kPa, preferably at most 200 kPa, more preferably at most 120 kPa. It will be clear to the skilled person to vary the temperature and pressure in relation to each other in order to achieve suitable conditions.

From the extractive distillation column, a stream including a first diol and possibly unsaturated hydrocarbons and/or one or more compounds with a carbonyl group and the extractant are removed as a bottom stream and a top stream including two or more diols are removed from the upper part of the column.

The top stream includes two or more C2 to C7 diols is removed from the first distillation column above the point at where the extractant is fed. In the separation of MEG and 1,2-BDO, this top stream would comprise 1,2-BDO; and in the separation of MPG and 2,3-pentanediol, this top stream would comprise 2,3-pentanediol. Preferably, the top stream is removed from the first distillation column as a condensed overheads stream. The top stream may contain other diols, such as MPG, 2,3-BDO, pentanediols, hexanediols and heptanediols. Preferably, this top stream is subjected to one or more fractional distillation steps in order to produce desired products as pure product streams.

The bottom stream from the first distillation column includes the first diol and possibly unsaturated hydrocarbons and/or one or more compounds with a carbonyl group and the extractant is sent to a second distillation column. The second distillation column produces the second stream as a top stream including a first diol, preferably MEG, and unsaturated hydrocarbons and/or one or more compounds with a carbonyl group and a bottoms stream including extractant. This distillation is preferably carried out at the same or lower pressure than in the extractive distillation step (in the first distillation column) in order to restrict the temperature in the reboiler and avoid or minimize potential product degradation. The distillation in the second distillation column is carried out at a temperature in the range of from 120 to 300° C., preferably from 150 to 250° C. Generally, the second distillation column is at a pressure of at least 1 kPa is preferred for economic reasons, with a pressure of at least 5 kPa more preferred for the same reasons. The pressure is at most 2000 kPa, preferably at most 200 kPa, more preferably at most 120 kPa. It will be clear to the skilled person to vary the temperature and pressure in relation to each other in order to achieve suitable conditions. In some embodiments in which the first diol is MEG, suitably, the diols content of this top stream, comprises at least 95 wt % MEG, preferably at least 98 wt % MEG, more preferably at least 99 wt % MEG, even more preferably at least 99.5 wt % MEG, most preferably at least 99.9 wt % MEG.

The third stream is removed as a bottoms stream from the second distillation is a used extractant stream. At least a portion of the used extractant stream may then be recycled to the first distillation column as at least a portion of the additional feed comprising an extractant. Any heavies left that had been present in the product stream including the three or more C2 to C6 diols may also be present in the extractant stream to be recycled. If the product stream including the three or more C2 to C6 diols is derived from the process for the hydrogenolysis of a carbohydrate-containing feedstock, such heavies are likely to be sugar alcohol like in their structure, boiling point and other physical properties and may be recycled with the rest of the extractant stream.

A portion of this used extractant stream may be removed as a bleed in order to prevent a build-up of heavies. In this embodiment, fresh extractant is provided to the first distillation column to make up the required amount of extractant. This fresh extractant should be provided to the first distillation column at the same height or above the used extractant stream. Optionally, at least a portion of this recycle stream may be subjected to further processing steps to further increase its purity.

After the separating step (ii), the second stream comprising a first diol and unsaturated hydrocarbons and/or one or more compounds with a carbonyl group is sent to the hydrogenation step (iii) disclosed above to remove any impurities, such as oxygenates, which might inhibit the first diol from passing the product specifications on color bodies and transmission of radiation.

In some embodiments, the hydrogenation step (iii) may be carried out by any suitable means known to one skilled in the art. In some embodiments, the hydrogenation reaction is carried out at a temperature within a range of about 20° C. to about 300° C. and a pressure within a range of about 0.5 bara to about 250 bara. In one arrangement, hydrogenation may be carried out in accordance with the process described in U.S. Pat. No. 6,137,016 which is incorporated herein by reference. The hydrogenation step provides a high purity diol stream, i.e., meets product specification on color bodies and transmission of UV radiation. For example, the standard test for ultraviolet transmittance of MEG is ASTM E2193. One skilled in the art would be able to determine the standard test for ultraviolet transmittance for other diols of interest.

High purity diol as used herein refers to a diol of at least 99 wt % purity, preferably at least 99.5 wt %, more preferably at least 99.6 wt % purity, most preferably at least 99.9 wt % purity. The on-spec diol stream will meet the product specifications on color bodies and transmission of radiation. Preferably, in the embodiment wherein the on-spec diol is MEG, the on-spec MEG is suitable for use as fibre grade MEG. Fibre grade MEG must meet transmittance at four different wavelengths (in nm) as shown in Table 1 below:

TABLE 1
Specification for Fibre grade MEG

| Wavelength (nm) | Transmittance (%) |
|---|---|
| 220 | 70 |
| 250 | 90 |
| 275 | 94 |
| 350 | 98 |

Any suitable hydrogenation catalyst may be used and could be determined by one of ordinary skill in the art. The hydrogenation catalyst is preferably an IUPAC update groups 7, 8, 9, 10, 11 metal-containing hydrogenation catalyst. Suitable IUPAC update groups 7, 8, 9, 10, 11 metal-containing catalysts typically contain from about 0.1 wt % up to about 2 wt % of an IUPAC update groups 7, 8, 9, 10, 11 or metals. Examples of IUPAC update groups 7, 8, 9, 10, 11 metals include nickel, palladium, platinum, rhodium, iridium, rhenium and the like, as well as mixtures of two or more thereof. The IUPAC update groups 7, 8, 9, 10, 11 metal or metals is, or are, deposited on an inert support, such as graphite, alumina, silica-alumina, silica, zirconia, thoria, a diatomaceous earth and the like. A particularly preferred catalyst is a nickel catalyst. This can contain, for example, from about 10 wt % up to about 60 wt % or more of nickel. Another is a palladium-on-carbon catalyst, preferably containing from about 0.1 wt % up to about 4 wt % of palladium.

Although the hydrogenation reaction can be conducted in the vapour phase, it is conveniently carried out as a liquid phase reaction, using either a slurry of the catalyst or, more preferably, a fixed bed of catalyst. When operating with a fixed bed of catalyst the catalyst particles preferably have a particle size in the range of from about 0.5 mm to about 5 mm. The particles may be of any convenient shape, such as spheres, pellets, rings or saddles.

When using a fixed bed of catalyst the reactor may be a shell-and-tube reactor, which can be operated isothermally. However, it is preferably an adiabatic reactor. The use of an adiabatic reactor is advantageous since its capital cost is much lower than that of a shell-and-tube reactor and it is generally much easier to charge with the chosen catalyst.

The hydrogenation reaction may be carried out at any suitable reaction conditions. In one arrangement, hydrogenation may be conducted at an elevated temperature of, for example, from about 30° C. to about 170° C. The feed temperature to the hydrogenation zone may be from about 50° C. to about 125° C. The hydrogenation may be carried out at an elevated pressure. Suitable pressures include those of, for example, from about 50 psia (about 3.45 bar) to about 2000 psia (about 137.90 bar), preferably from about 150 psia (about 10.34 bar) up to about 1000 psia (about 68.95 bar).

The second stream including a first diol and unsaturated hydrocarbons and/or one or more compounds with a carbonyl group stream may be supplied to the hydrogenation reactor at a liquid hourly space velocity of from about 0.1 h−1 to about 4.0 h−1, preferably from about 0.5 h−1 to about 1.5 h−1.

An inert diluent may be mixed with the feed prior to entering the hydrogenation zone. In one arrangement, the inert diluent may be a recycle from the exit from the hydrogenation zone. In this arrangement, the ratio of inert diluent to fresh feed preferably lies in the range of from about 1:1 to about 1000:1.

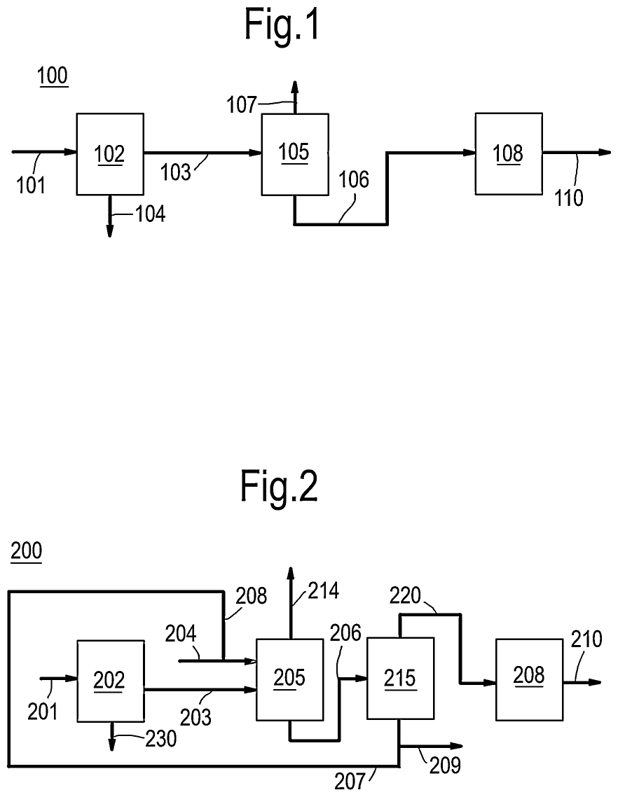

Turning now to the Figures, FIG. 1 illustrates a block flow diagram of an embodiment of a process of the present invention. The process 100 for the separation of a diol from a product stream includes providing a product stream 101 having three or more C2 to C6 diols, C3 to C6 sugar alcohols and C4 to C6 polyhydric alcohols with at least 3 hydroxyl groups in the molecule, and a catalyst as a feed to a first separation unit 102. The separation unit 102 may either be a flash unit or a distillation column. The separation unit 102 produces a heavies stream 104 and a first stream 103 comprising the three or more C2 to C6 diols and possibly unsaturated hydrocarbons and/or one or more compounds with a carbonyl group from the product stream 101.

The first stream 103 is sent to a distillation unit 105 to produce a second stream 106 including a first diol and unsaturated hydrocarbons and/or one or more compounds with a carbonyl group and a third stream 107 including two or more diols. In some embodiments, the distillation unit 105 may be one or two distillation columns.

The second stream 106 is sent to a hydrogenation unit 108 the second stream comprising a first diol and unsaturated hydrocarbons and/or one or more compounds with a carbonyl group to provide a high purity diol stream 110.

FIG. 2 illustrates a block flow diagram of an alternative embodiment of a process of the present invention. The process 200 for the separation of a diol from a product stream includes providing a product stream 201 having three or more C2 to C6 diols, C3 to C6 sugar alcohols and C4 to C6 polyhydric alcohols with at least 3 hydroxyl groups in the molecule, and a catalyst as a feed to a first separation unit 202. The separation unit 202 may either be a flash unit or a distillation column. The separation unit 202 produces a heavies stream 230 and a first stream 203 comprising the three or more C2 to C6 diols and possibly unsaturated hydrocarbons and/or one or more compounds with a carbonyl group from the product stream 201.

The first stream 203 is sent to a first distillation unit 205 to produce a second stream 206 including a first diol and unsaturated hydrocarbons and/or one or more compounds with a carbonyl group and a third stream 214 including two or more diols.

An extractant stream 204 is also provided to the first distillation column 205 at the same height or above the first stream 203. In some embodiments, the first distillation 205 column operates at a temperature in the range of from 100 to 300° C. and a pressure in the range of from 0.1 to 2000 kPa. The bottoms stream 206 is subjected to distillation in a second distillation column 215, which is operated to provide a first diol stream as an overheads stream 220. The remaining extractant is removed as a bottoms stream 207 and can be recycled to provide the used extractant 208 to the first distillation column 205. A bleed stream 209 is removed from the extractant recycle stream in order to prevent a build-up of heavies.

The overheads stream 220 including a first diol and unsaturated hydrocarbons and/or one or more compounds with a carbonyl group is supplied to a hydrogenation reactor 208 to provide a high purity diol stream 210.

### EXAMPLES

Embodiments will be further illustrated by the following, non-limiting examples.

### Comparative Example 1—Mixed Glycols Isolation and Extractive Distillation

Glycol mixtures were obtained by conversion of glucose as described in WO2018/064245, the entire disclosure of which is hereby incorporated by reference. A total of 165.2 kg reactor effluent was obtained from a reactor feed including 19.6 kg glucose and 145.6 kg water in total.

Water and light components like traces of methanol and ethanol were removed in ten separate batches by rotary evaporation, which mimics flashing. A liquid fraction, mostly water, of 144.9 kg was collected and discarded leaving an organic fraction.

A mixed glycols fraction of 16.9 kg was obtained by subsequent rotary evaporation of the organic fraction. Mass balances of the ten individual batches indicate a loss of about 1.7 kg glycols with the water fraction, while the water content in the mixed glycols fraction could be as high as 2.4 kg water. These mass balances indicate a total yield of 14.5 kg of glycols recovered from the 16.9 kg mixed glycols fraction obtained (89.5 % w of total mixed glycols generated).

Also obtained from the subsequent rotary evaporation of the mixed glycols fraction is a residual fraction of 3.4 kg comprising sorbitol, erythritol, glycerol and residual catalyst. The residual fraction has not been analyzed in further detail.

TABLE 1
Mass balance of mixed glycols preparation and isolation

| | | Rotary | Rotary | Rotary |
|---|---|---|---|---|
| | | evaporation | evaporation | evaporation |
| | Reactor | water fraction | mixed glycols | heavy residue |
| | feed (kg) | (kg) | (kg) | (kg) |
| Glucose | 19.6 | | | |
| Glycols | | 1.7 | 14.5 | |
| Water | 145.6 | 143.2 | 2.4 | |
| Polyols | | | | 3.4 |

The composition of the mixed glycols fraction has been analyzed by GC analysis, while the glycerol fraction was measured by LC analysis (Table 2—Feed). This mixed glycols fraction was used as feed for extractive distillation.

A first 2-inch glass double-wall distillation column was used for extractive distillation, with glycerol as extractant. The first column had three sections of approximately 167 cm height each. The top section was empty, while the middle and bottom sections were filled with Sulzer Mellapak Y-500 Hastelloy, approximately 140 cm total height each. The feed position was at ⅔ from the top, in between the two packed sections. The extractant feed entry was from the top of the first column. Height equivalent of a theoretical plate (HETP) was estimated at 22 cm.

A second 2-inch glass double-wall distillation column was used for ethylene glycol recovery and extractant recycling. The second column also had three sections of approximately 167 cm height each. The top section was empty, while the middle and bottom sections were equipped with Sigma-Aldrich Pro-Pak distillation packing. The middle section packing has a height of 20 cm, while the packing height of the bottom was 10 cm. The feed position was at ⅔ from the top, in between the two packed sections. HETP was estimated at 22 cm.

The first distillation column was operated at 231 mbar pressure, measured at the top of the column, a condenser temperature of 132° C. and a reboiler liquid temperature of 180° C. The mixed glycols feed flow rate was 50 g/h and the glycerol feed flow rate was 130 g/h, resulting in a top product flow rate of 6 g/h and a bottom product flow rate of 178 g/h. Water was fed into the reboiler at a flow rate of 0.5 g/h. The top reflux flow rate was gradually reduced over time, from 350 g/h to 32 g/h, representing a gradual decline in reflux ratio.

The second distillation column was operated at 91 mbar pressure, measured at the top of the column, a condenser temperature of 125° C. and a reboiler liquid temperature of 202° C. The feed to the second distillation column is the bottom product of the first distillation column, at a flow rate of 178 g/h, resulting in a top flow rate of 43 g/h and a bottom extractant flow rate of 134 g/h. Water was fed into the reboiler at a flow rate of 0.5 g/h. The top reflux flow rate was 25 g/h.

The compositions of the top product in the first distillation column and the top product of the second distillation column is given in Table 2.

TABLE 2
Compositions from Example 1

| | Feed | Top C-1 | Top C-2 |
|---|---|---|---|
| Component | [g/kg] | [g/kg] | [g/kg] |
| Ethylene glycol | 808.7 | 364.4 | 994.7 |
| Propylene glycol | 46.7 | 446.9 | 0.0 |
| 1,2-butanediol | 31.1 | 186.0 | 0.0 |
| 1,2-hexanediol | 10.6 | 0.0 | 6.5 |
| 2,3-pentanediol | 7.5 | 14.1 | 0.0 |
| isomers | | | |
| 2,3-butanediol | 4.2 | 16.7 | 0.0 |
| isomers | | | |
| x,y-hexanediol | 2.8 | 5.0 | 0.0 |
| isomers | | | |
| Cyclic diol 1 | 2.3 | 3.5 | 0.0 |
| glycerol | 2.0 | 0.0 | 0.0 |
| 2,5-hexanediol | 1.9 | 0.0 | <0.5 |
| 1,2-pentanediol | 1.8 | 2.7 | 0.0 |
| Cyclic diol 2 | 1.5 | 2.5 | 0.0 |
| Isosorbide | 1.3 | 0.0 | 0.0 |
| Total | 922 | 1042 | 998 |

The UV transparency of the MEG obtained was measured according to ASTM E2193, Standard Test Method for Ultraviolet Transmittance of Monoethylene Glycol (using Ultraviolet Spectrophotometry) applying a Perkin Elmer Lambda 35 UV-Vis Spectrometer (serial nr.502S10121302). Sales specifications for UV transmission are 70% (220 nm); 90% (250 nm); 94% (275 nm) and 98% (350 nm). Measured UV transmissions are 54% (220 nm); 82% (250 nm); 82% (275 nm) and 97% (350 nm). The required sales specification for UV transmission was not reached thus the top stream from C-2 is sent for further processing.

### Example 2 Mixed Glycols Isolation and Extractive Distillation/Hydrogenation

To provide a MEG sample which closely resembles the off-spec product of Comparative Example 1, commercially available fossil-based high-purity MEG (fiber grade) was evaluated for UV transparency according to ASTM E2193 and measured UV transmissions are 90% (220 nm); 98% (250 nm); 99% (275 nm) and 100% (350 nm). This MEG sample was subjected to extractive distillation as described in Comparative Example 1. The UV transparency of the MEG obtained has been measured according to ASTM E2193. Measured UV transmissions are 64% (220 nm); 82% (250 nm); 82% (275 nm) and 99% (350 nm). The UV transmissions obtained closely matches the UV transmissions of the MEG sample obtained after extractive distillation in Comparative Experiment 1. While not being bound by theory, this may indicate that the deviation in UV transparency relative to high-purity MEG is mainly due to the presence of components formed during the extractive distillation process, possibly by thermal radiation of glycerol and/or MEG. No indication of significant contamination by components originating from the conversion of saccharides to glycols is apparent.

The MEG sample obtained after extractive distillation was subjected to hydrogenation. A Hastelloy 250 ml magnetically stirred Parr autoclave was loaded with 85.92 g MEG, to which a slurry of 2.02 g Raney Nickel 2800 (Aldrich) and 12.12 g water was added. The autoclave was closed, flushed three times with nitrogen and three times with hydrogen, after which the temperature was raised to 119° C. for 120 min. The total pressure was maintained at 77 barg by adjustment of the hydrogen pressure.

The liquid obtained was centrifuged to remove solid particles. The UV transparency of the liquid was measured according to ASTM E2193. Measured UV transmissions are 91% (220 nm); 99% (250 nm); 99% (275 nm) and 100% (350 nm).

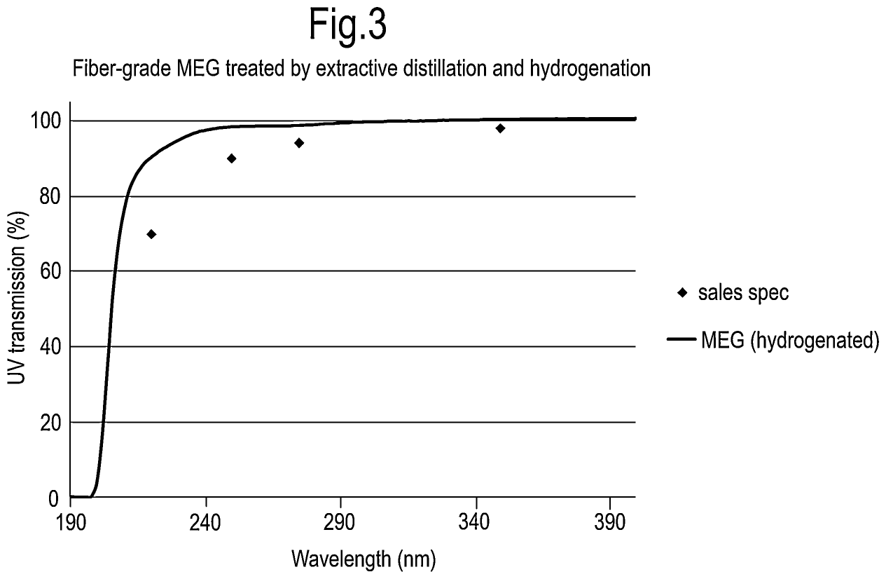

No attempt was made to remove the water, originating from the catalyst slurry. The UV spectrum obtained was corrected mathematically for the presence of water to obtain UV transmissions for MEG, assuming water to be fully transparent over the UV range measured. This mathematical correction results in a slightly lower UV transparency and has been verified in separate experiments by dilution of MEG with water (results not shown). Corrected UV transmissions for MEG are 90% (220 nm); 98% (250 nm); 99% (275 nm) and 100% (350 nm) (FIG. 3).

The MEG obtained after hydrogenation meets the UV sales specifications. As shown in FIG. 3, the trials described above meet or exceed the transmission percentage acceptable for fibre grade MEG.

The present invention has a number of advantages over prior art processes, wherein problems are encountered with diol products not meeting the UV specification for fibre grade polymers and/or fibres. Firstly, heavy (high-boiling) by-products are removed by distillation in a first distillation column. Then, in a second distillation column, one or more extractants are used for the selective extractive distillation of the first diol. The strong interaction between the sugar alcohols and the first diol breaks any azeotrope and affects the volatility of the diols present, allowing them to be separated. A simple distillation of the first diol as overhead product from the extractant in a third distillation column results in a stream including a first diol and unsaturated hydrocarbons and/or one or more compounds with a carbonyl group, meeting the UV sales specifications after hydrogenation, providing a high purity first diol stream, for example high purity MEG suitable for use as fibre grade MEG either immediately or after removal of trace compounds.

Although a few embodiments of the disclosure have been described in detail above, those of ordinary skill in the art will readily appreciate that many modifications are possible without materially departing from the teachings of this disclosure. Accordingly, such modifications are intended to be included within the scope of this disclosure as defined in the claims.

1. A process for the separation of a diol from a product stream, said process comprising the steps of:
(i) separating the product stream comprising three or more C2 to C6 diols, C3 to C6 sugar alcohols, and C4 to C6 polyhydric alcohols with at least 3 hydroxyl groups in the molecule, and a catalyst, to produce a first stream comprising the three or more C2 to C6 diols;
(ii) separating the first stream comprising the three or more C2 to C6 diols into
a) a second stream comprising a first diol and unsaturated hydrocarbons and/or one or more compounds with a carbonyl group, and
b) a third stream comprising two or more diols;
(iii) hydrogenating the second stream comprising a first diol and unsaturated hydrocarbons and/or one or more compounds with a carbonyl group to provide a purified diol stream.

2. The process of claim 1, wherein the product stream is, or is derived from, a product stream of a carbohydrate hydrogenolysis process, and wherein the product stream may further comprise oxygenate impurities.

3. The process of claim 1 or 2, wherein the separating of step (i) is evaporating, preferably evaporating performed in a distillation unit or in a flash unit.

4. The process of any one of claims 1 to 3, wherein the separating of the first stream of step (ii) comprises extractive distillation using an extractant.

5. The process of claim 4, wherein the extractant is selected from the group of C3 to C6 sugar alcohols, C4 to C6 polyhydridic alcohols with at least 3 hydroxyl groups in the molecule, and mixtures thereof.

6. The process of claim 4 or 5, wherein the extractant comprises glycerol.

7. The process of any one of claims 4 to 6, wherein the extractive distillation comprises:
extracting an intermediate stream comprising a first diol and possibly unsaturated hydrocarbons and/or one or more compounds with a carbonyl group from the first stream using an extractant fed above the first stream at a temperature in the range of from 100 to 300° C. and a pressure in the range of from 0.1 to 2000 kPa; and
distilling the intermediate stream to produce the second stream.

8. The process of any of claims 4 to 7, wherein the extractant is added in an amount such that the weight ratio of the extractant to the first stream is at least 1:2 and at most 20:1 based on the overall weight of the feed/mixture.

9. The process of any one of claims 1 to 3, wherein the separating of the first stream of step (ii) comprises distilling the first stream to produce the second stream and the third stream at a temperature in the range of from 100 to 300° C. and a pressure in the range of from 0.1 to 2000 kPa.

10. The process of claim 9, wherein the third stream comprises an azeotrope of MEG and 1,2-BDO or an azeotrope of MPG and 2,3 BDO.

11. The process according to any one of claims 1 to 10, wherein the hydrogenating of step (iii) comprises a hydrogenation reaction carried out at a temperature within a range of about 20° C. to about 300° C. and a pressure within a range of about 0.5 bara to about 250 bara in the presence of a hydrogenation catalyst.
